(19) United States
(12) Patent Application Publication
(10) Pub. No.: US 2018/0133422 A1
(43) Pub. Date: May 17, 2018

(54) DEVICES THAT ALLOW FOR RAPID CHANGE OUT OF ENDOTRACHEAL (ET) TUBES AND RELATED METHODS

(71) Applicant: Wake Forest University Health Sciences, Winston-Salem, NC (US)

(72) Inventors: Michael A. Olympio, Winston-Salem, NC (US); Philip J. Brown, Winston-Salem, NC (US)

(21) Appl. No.: 15/578,453

(22) PCT Filed: Jun. 16, 2015
(86) PCT No.: PCT/US2015/035953

§ 371 Date: Nov. 30, 2017

(51) Int. Cl. A61M 16/04; A61B 1/267

(52) U.S. Cl. CPC A61M 16/049; A61M 2025/0183; A61B 1/267

(57) ABSTRACT

Tracheal tube devices include: (a) a mouthpiece sized and configured to allow an endotracheal tube to extend outwardly therefrom; and (b) a handle holding a cutting member. The mouthpiece can include at least one outwardly projecting substantially rigid short tube. The cutting member may be held in a handle releasably attached to the short tube.

### FIELD OF THE INVENTION

The present invention relates to medical devices.

### BACKGROUND

Endotracheal (ET) tubes are used to protect a patient's airway can and can sometimes be difficult to insert. An ET tube can be connected to a ventilator to help the patient breathe. Often, once the ET tube is in position, it remains in position and holds the patient's airway open. If the ET tube is prematurely removed or exchanged under adverse conditions, the airway can swell shut or become difficult to re-secure. Unfortunately, over time, the ET tube can be a pathway for bacteria or other undesired pathogens or may become somewhat occluded by biofilms or mucus or components. Also, the tube itself may undesirably change, e.g., the cuff may fail or the tube may change in size and/or shape which might require changing the tube, again sometimes under difficult circumstances.

### SUMMARY OF EMBODIMENTS OF THE INVENTION

Embodiments of the present invention allow an endotracheal tube to be replaced (changed out) with stability while maintaining the patency of an airway under direct visualization of the internal trachea.

Embodiments of the invention are directed to tracheal tube devices. The devices include a mouthpiece with an external port merging into an open internal channel sized and configured to allow an endotracheal tube to extend therethrough into a trachea of a subject. The external port has a perimeter. The mouthpiece also includes at least one short tube with a cavity residing above the external port. The device also includes a handle with at least one interlock member. In an operative configuration, the handle extends laterally away from the mouthpiece to position a distal end thereof at between 3-10 inches away from the mouthpiece. The interlock member interlocks to the at least one short tube and comprises a cutting blade that extends inward from a respective short tube to be positioned above but inside the perimeter of the external port. An outerwall of the short tube defines a bounding surface of the perimeter of the external port and/or the short tube resides adjacent a medial bottom segment of the external port.

The external port can have an elongate configuration with a width dimension greater than a length dimension. The short tube can reside proximate a medial segment of the width dimension. In use, the handle can be held by the short tube to be able to selectively and laterally extend to either a right or left side of a patient or in a caudal orientation to the patient.

The short tube can reside adjacent the medial bottom segment of the external port.

The at least one interlock member can include a primary interlock member that projects a distance of between 0.25 inches and 4 inches below a bottom surface of the handle which can be slidably receivable into the cavity of the short tube. The cavity of the short tube can have a depth that is between about 0.25 inches and 4 inches.

The short tube can have an outwardly extending wall that tapers inwardly in a direction toward the external port of the mouthpiece into a smaller size.

The primary interlock member can include first and second cooperating semicircular halves, the first half can be attached to the first handle member and the second half can be attached to the second handle member. Each of the cooperating semicircular halves can have flat inner surfaces that face each other.

The handle can hold a metallic band that defines the cutting blade. The metallic band can have a substantially cylindrical longitudinally extending segment. In an operative configuration, the handle can extend laterally away from the mouthpiece while the at least one interlock member resides in the short tube with the cylindrical segment held over the external port of the mouthpiece a distance in front of the short tube.

The substantially cylindrical segment can have a fenestrated configuration.

The handle can include first and second cooperating handle members that can open and close relative to each other and that hold a metallic member defining the cutting blade therebetween. The handle can also include a pivotable latch member residing on one of the first and second members configured to latch and unlatch the first and second members.

The first and second handle members can include an outer upper surface, each can have aligned raised pads. The latch can have a window that encases the aligned raised pads in a latched configuration.

The at least one short tube can be a single short tube. The short tube can have an outer rim with notches. The at least one handle interlock member can include a lower surface with a circumferentially extending groove and radially extending teeth. The groove can be configured to abut the rim of the short tube and the teeth are configured to reside in respective notches.

The notches can have an open “V” shape with a larger end facing upward and the teeth have a V shape with a smaller end facing downward.

The cutting blade can be configured as a malleable unitary surgical metallic band that wraps together to define a (longitudinally extending) cylindrical channel and defines a cutting edge.

Yet other embodiments are directed to medical mouthpieces. The mouthpieces include an external port merging into an open internal channel sized and configured to allow an endotracheal tube to extend therethrough. The external port has a perimeter. The mouthpiece also includes at least one short tube with a cavity residing above the external port. The mouthpiece has a monolithic molded body. An outerwall of the short tube defines a bounding surface of the perimeter of the external port and/or the short tube resides adjacent a medial bottom segment of the external port.

The external port can have an elongate configuration with a width dimension greater than a length dimension. The short tube can reside proximate a medial segment of the width dimension.

The cavity of the short tube can have a depth that is between about 0.25 inches and 4 inches.

The short tube can have an outwardly extending wall that tapers inwardly in a direction toward the external port of the mouthpiece into a smaller size.

The at least one short tube can be a single short tube. The short tube can have an outer rim with circumferentially spaced apart notches.

Still other embodiments are directed to medical devices that include a handle. The handle includes first and second cooperating, laterally extending handle members with a length between 3-10 inches that can open and close relative to each other. The handle holding a metallic band defining a cutting blade therebetween. The metallic band has a (longitudinally extending) cylindrical segment that resides outside the first and second handle members. The handle comprises an interlock member that projects a distance of between 0.25 inches and 4 inches below the handle.

The device can include a pivotable latch member residing on one of the first and second members configured to latch and unlatch the first and second members.

The interlock member can include first and second cooperating semicircular halves, a first half can be attached to the first handle member and the second half can be attached to the second handle member. Each of the cooperating semicircular halves can have flat inner surfaces that face each other.

The first and second handle members can have an outer upper surface, each with aligned raised pads, and the latch can have a window that encases the aligned raised pads in the latched configuration.

The handle can have a lower surface with a circumferentially extending groove extending about the interlock member and radially extending teeth that extend inward from the groove.

The teeth can have a V shape with a smaller end facing downward.

The metallic band can have a fenestrated configuration.

Still other embodiments are directed to medical kits that include any of the aforementioned devices (e.g., handle, mouthpiece) with any of the described features, in combination with a clamp, all held in at least one sterile package.

Still other embodiments are directed to methods of changing respective endotracheal tubes. The methods include: (a) cutting an exposed portion of an endotracheal (ET) tube extending out of patient at an angle; then (b) pulling the endotracheal (ET) tube out of patient through a mouthpiece while the mouthpiece is in position on a patient with an endoscope extending therethrough; (c) cutting a slit in the wall of the ET tube based on the pulling step using a cutting blade interlocked to the mouthpiece by an interlock member of a handle comprising a downwardly extending projection held in a cavity of a short tube proximate the ET tube port of the mouthpiece; (d) removing the ET tube from the patient; then (e) inserting a different ET tube into the patient over the endoscope after the cutting while the mouthpiece remains on the patient, allowing change out of the ET tube while (i) maintaining visualization through the endoscope of a carina at a distal end of the trachea that splits to right and left lung bronchi and concurrently (ii) maintaining direct access within the trachea during the exchange process to thereby provide a clinician reassurance that access and pathway will not be compromised during the exchange and concurrently (iii) providing stability of the entire exchange device, endoscope and ET tube.

The cutting member can be defined by a malleable surgical metal band that wraps together to define a cylindrical channel. The method can include placing the metal band about an outerwall of an endoscope so that the endoscope resides snugly in the cylindrical channel.

The device can also be called an “endotracheal tube exchanger” that can be used in conjunction with a fiber optic bronchoscope (i.e., endoscope) and can allow a physician to (a) directly maintain visualization of the internal airway as the old tube is cut and removed, (b) slide the new endotracheal tube into place quickly and confidently, (c) maintain stability and avoid injury, (d) optimize depth of the new tube, and (e) deliver oxygen substantially or even entirely throughout the entire exchange.

The entire device can be single-use disposable. In other embodiments, the cutting member is single-use disposable and the handle can be sterilized and re-used.

The device can include a slicing tool handle that (a) securely fastens the fiber optic scope for the slicing/cutting of the endotracheal tube being removed and includes an oral (mouthpiece) interlocking stabilizer that protects the patient and physician against the force of cutting and exchange.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner These and other objects and/or aspects of the present invention are explained in detail in the specification set forth below.

Other systems and/or methods according to embodiments of the invention will be or become apparent to one with skill in the art upon review of the following drawings and detailed description. It is intended that all such additional systems, methods, and/or devices be included within this description, be within the scope of the present invention, and be protected by the accompanying claims.

### BRIEF DESCRIPTION OF THE DRAWINGS

Other features of the present invention will be more readily understood from the following detailed description of exemplary embodiments thereof when read in conjunction with the accompanying drawings.

FIG. 1 is a side perspective view of another embodiment of the device in position on a (phantom) patient according to embodiments of the present invention.

FIG. 2A is a top, side perspective view of the cutting member in cooperating alignment for attachment to a short tube of the mouthpiece according to embodiments of the present invention.

FIG. 2B is an enlarged top perspective view of the device shown in FIG. 2A.

FIG. 2C is a caudal, bottom perspective view of the device shown in FIG. 2A.

FIG. 2D is an enlarged caudal, bottom, side perspective view of the device shown in FIG. 2A.

FIG. 2E is a side perspective view of the device shown in FIG. 2A but illustrating the mouthpiece in a visually transmissive configuration to illustrate an exemplary nesting of the mouthpiece engagement end of the handle according to some embodiments of the present invention.

FIG. 3 is a side, bottom perspective view of the handle according to embodiments of the present invention.

FIG. 4 is a hemi-section view of the handle and mouthpiece of the device shown in FIG. 3.

FIG. 5 is a bottom, side perspective view of one handle member of the handle shown in FIGS. 4 according to some embodiments of the present invention.

FIG. 6A is a top perspective view of another embodiment of a device for endotracheal tube changeout with a pivoting clasp according to embodiments of the present invention.

FIG. 6B is an enlarged top perspective view of the device shown in FIG. 6A with the clasp in the lock orientation according to embodiments of the present invention.

FIG. 7A is a front, side perspective view of the device shown in FIG. 6A, shown in an open or partially open (delatched) configuration according to embodiments of the present invention.

FIGS. 7B and 7C are front, side perspective views of exemplary open (delatched) configurations of the handle with the cutting/slicing blade according to embodiments of the present invention.

FIG. 7D is a front perspective view of the exemplary latched configuration of the handle shown in FIG. 7C with the cutting/slicing blade according to embodiments of the present invention.

FIG. 7E illustrates the handle shown in FIG. 7D with the blade closed against the fiberoptic (endo/broncho)scope according to embodiments of the present invention.

FIG. 8A is an enlarged top perspective view of a mouthpiece according to some embodiments of the present invention.

FIG. 8B is a greatly enlarged side perspective view of the mouthpiece shown in FIG. 8A according to embodiments of the present invention.

FIG. 8C is a greatly enlarged opposing side perspective view of the mouthpiece shown in FIG. 8B.

FIG. 8D is a top, cephalad view of the device shown in FIG. 8A.

FIG. 9 is an exemplary set of medical devices that can be provided in a ready-to-use kit to facilitate a rapid endotracheal tube changeout according to embodiments of the present invention.

### DETAILED DESCRIPTION OF EMBODIMENTS OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise. One or more features shown and discussed with respect to one embodiment may be included in another embodiment even if not explicitly described or shown with another embodiment. The abbreviations of “FIG.” and “Fig.” are used interchangeably with the word “Figure” in the application and drawings.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “caudal”, “cephalad”, “inferior” and “superior” and the like are used herein for the purpose of explanation only unless specifically indicated otherwise.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise. In the claims, the word “a” with respect to an element is intended to include one or more of such elements and is not limited to a single such element unless stated otherwise.

The term “about” means that the recited number or value can vary by+/−20%.

The term “sterile” means that the noted device or material meets or exceeds defined medical guidelines of cleanliness and is substantially (if not totally) without contaminants so as to be suitable for medical uses.

The term “short tube” refers to a tube attached or integral to a mouthpiece that has a length that is between about 0.25 inches to about 4 inches, more typically between about 1 to about 2.5 inches.

Embodiments of the invention are particularly suitable for human or animal use.

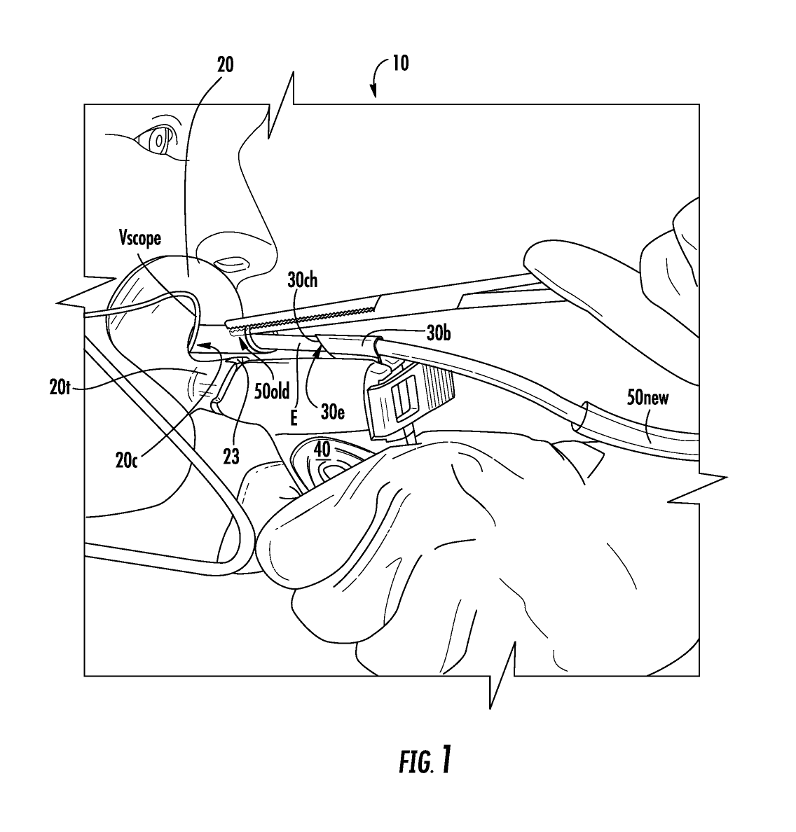

Turning now to the figures, FIG. 1 illustrates one embodiment of a tracheal assembly 10. As shown, the tracheal assembly 10 includes a mouthpiece 20. The mouthpiece 20 defines at least one access channel 20c that can slidably receive an endotracheal (ET) tube 50. The device 10 includes a cutting blade 30 that is sized and configured to cut into an outer wall 50w of the ET tube 50 proximate the mouthpiece 20 as the tube 50 is slidably removed from a patient. The cutting blade 30 can have any suitable configuration but typically has a single or primary sharp edge 30e that faces the mouthpiece 20 and may optionally comprise a flat razor or scalpel. The term “cutting blade” is used interchangeably with the term “cutting member” and is used broadly to include any member having at least one cutting edge or surface. Other characteristics of the cutting blade 30 may include one or more of an appropriate tensile strength, shape memory material, razor-sharpness, stiffness, resistance to twist or torque and the like.

FIG. 1 also illustrates that the indwelling endotracheal tube 50old can be cut at an angle to define a leading end at a position that is proximate but above the mouthpiece 20, at initiation or just prior to start the removal process.

Typically, the mouthpiece channel 20c concurrently receives both the ET tube 50 and an endoscope E (i.e., “bronchoscope”) that extends into the trachea of the patient within the ET tube. The cutting blade 30 orients its cutting edge 30e over the channel 20c to be able to cut into a wall of the adjacent ET tube 50 as the ET tube is pulled from the patient over the endoscope E.

The endoscope E can (indeed should) remain in position in the patient during the change out of the ET tubes. As shown in FIG. 1, a new ET tube 50new can reside on the endoscope E above the “old” ET tube 50old that is being replaced so that the new tube 50new can be slid down over the endoscope E (which acts as a stylette or guidewire), typically after the handle 40 (where used) is at least partially removed so as to not occlude the travel path to position the new tube properly in the patient once the old tube 50old has been removed from the patient. FIG. 1 illustrates that a viewing scope lead (Vscope) can also be in position during the procedure.

FIGS. 1, 2A-2E, 3 and 4, for example, show that the device 10 can include a handle 40 that can engage the mouthpiece 20.

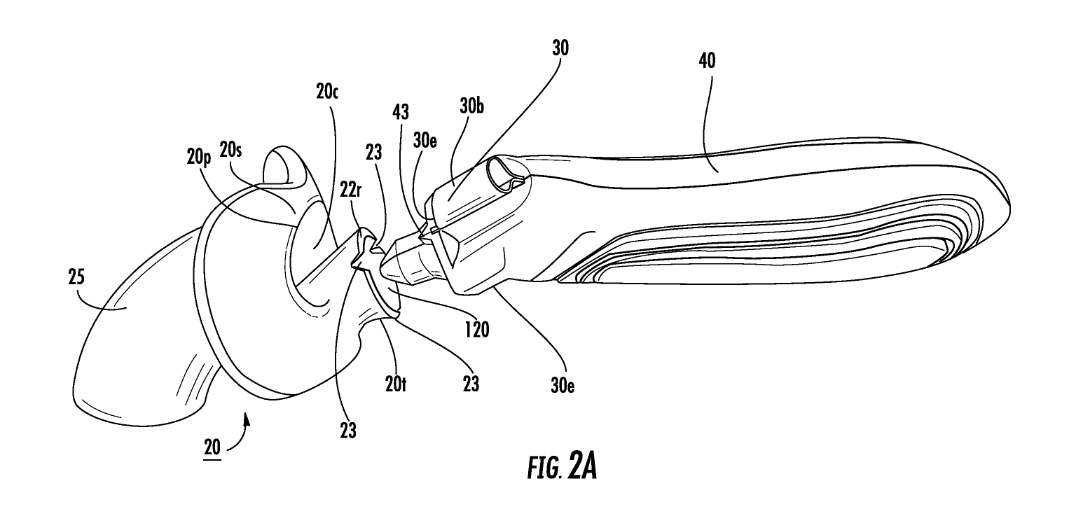

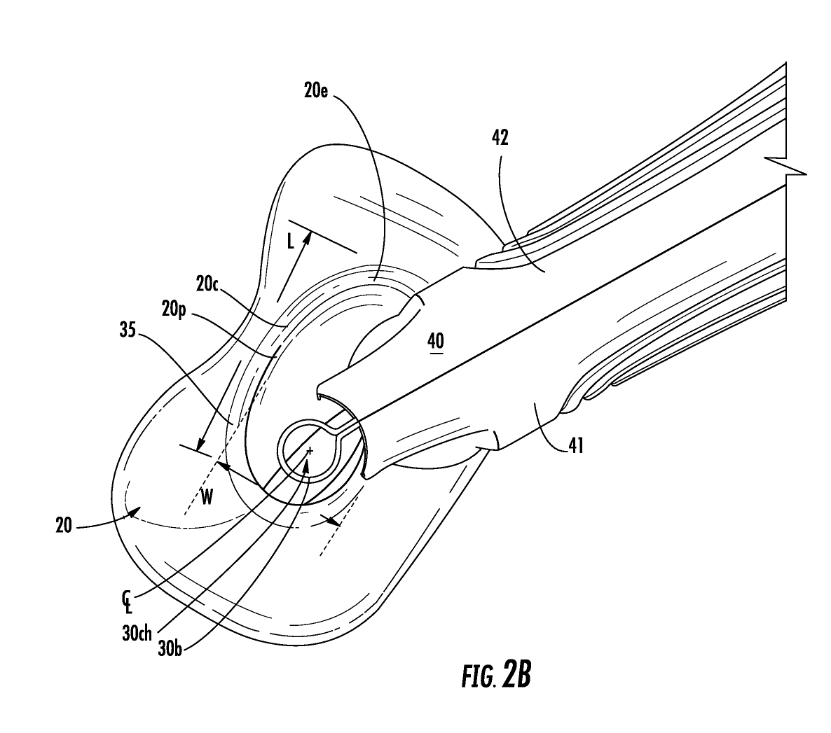

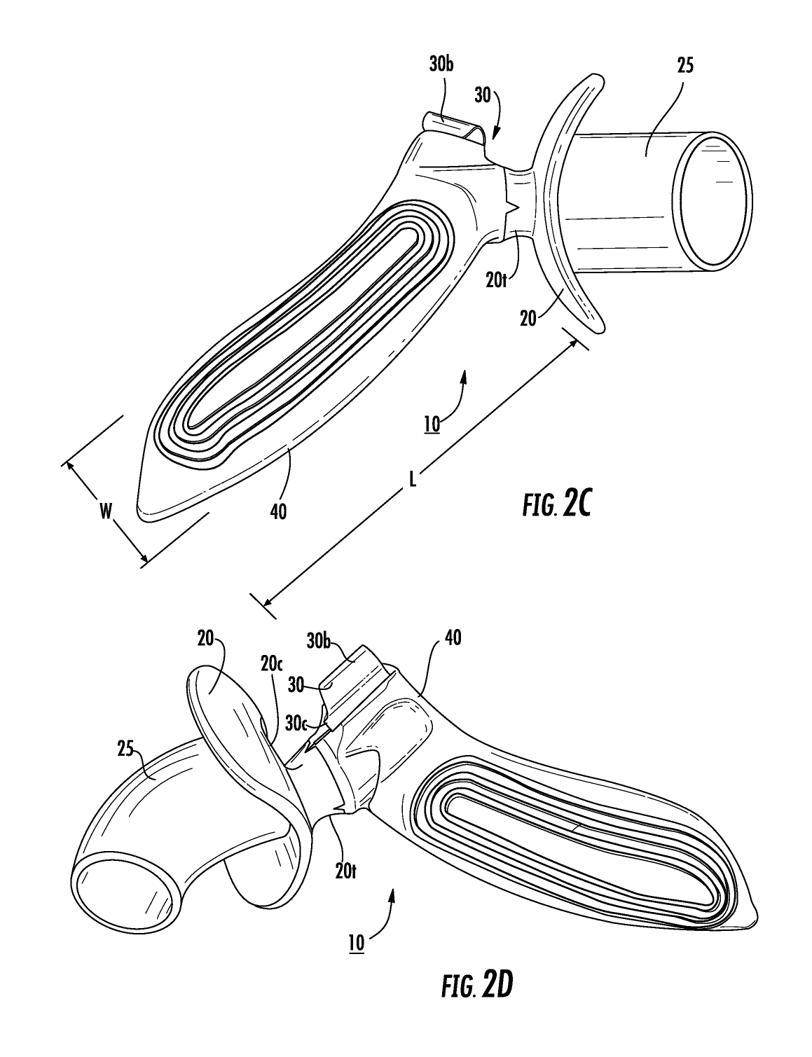

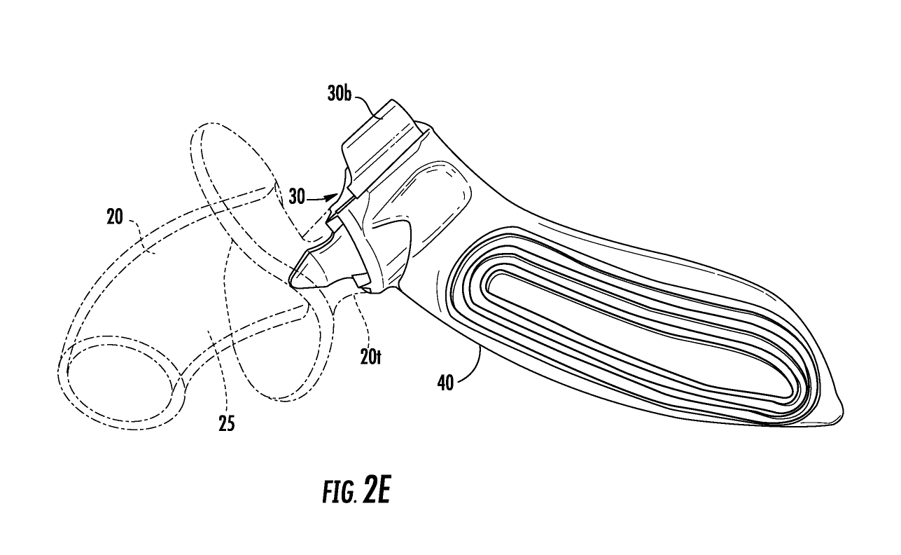

The handle 40 is typically configured to releasably engage the mouthpiece 20. Referring to FIGS. 2A, 2C-2E, the handle 40 can have any suitable laterally extending (in a direction away from the mouthpiece) length “L” but is typically between 2-10 inches, more typically between 3-10 inches, such as about 3 inches, about 4 inches, about 5 inches, about 6 inches, about 7 inches, about 8 inches, about 9 inches or about 10 inches. The handle 40 can have a maximal width “W” that is between about 0.25 inches to about 2 inches, typically about 1 inch.

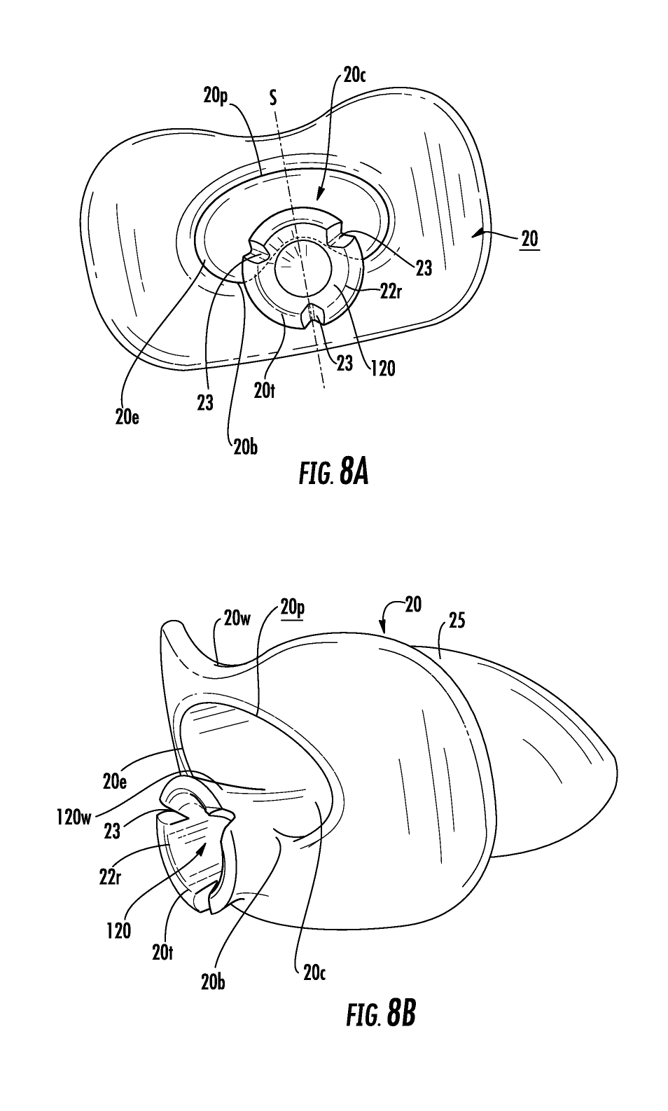

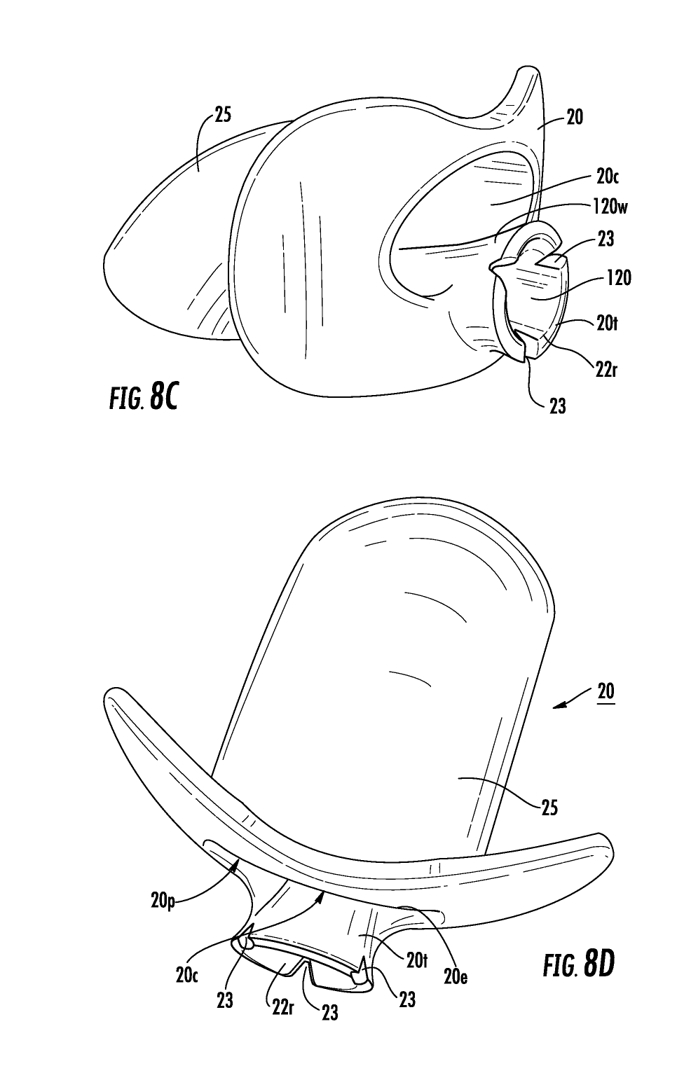

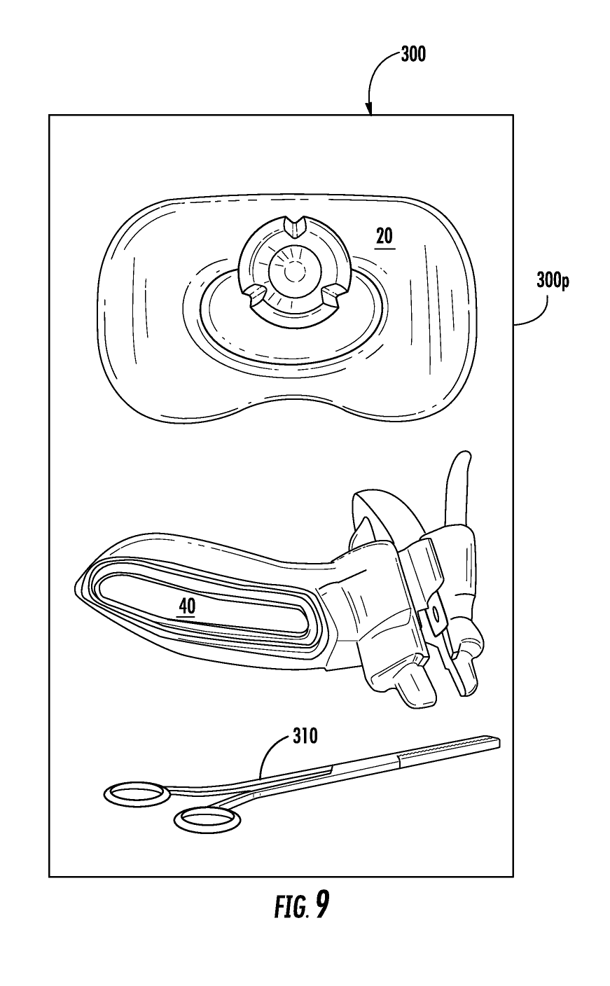

The mouthpiece 20 can have a substantially rigid or semi-rigid monolithic body. The mouthpiece 20 can alternately comprise cooperating components that snap or otherwise attach together or can be separated apart, such as along a split line S as shown in FIG. 8A. The split line S can extend over an external port/lumen 20e that merges into the internal channel 20c and may also extend over the short tube 20t to provide a seam for easy installation or removal about a respective ET tube 50 thereat.

As shown in FIG. 1, in operative use, a user can manually push down on the handle 40 to apply a force against the mouthpiece 20 while the ET tube 50old is pulled out. The ET tube 50 is typically pulled out by the same (or a different) clinician (e.g., doctor or nurse) applying the extraction force. The person withdrawing the old tube 50old can use fingers and/or a clamp 310 (e.g., a Kocher clamp, hemostat or forceps) to attach to and pull the “old” ET tube 50old with an upward force direction to slit the outer wall of the respective tube 50old using the cutting blade 30 to remove the old tube 50 while the endoscope E remains in position. The term “Kocher clamp” refers to a rigid, straight hemostat with interlocking teeth on the tip. The person removing the tube 50 can use a pull to a right or left direction. The handle 40 can be inserted into the short tube 20t to be able to extend in different lateral or medial/cephalad directions depending on physician preference or access to the patient.

As shown in FIGS. 2B, 3, 6A, 6B, and 7A-7C, the handle 40 can have first and second matably attachable elongate members 41, 42 that can attach together before or after one or both are attached to the mouthpiece 20. In other configurations, the handle 40 can have a single piece body.

The mouthpiece 20 can be configured for use as an ET tube holder that can include a locking clip for secure tube position and/or as a modified conventional biteblock 25 for endoscopy. Thus, the mouthpiece 20 can be placed on the patient during the initial ET tube insertion procedure. The handle 40 and mouthpiece 20 can be used to stabilize an endoscope during other endoscopic procedures such as, for example, a bronchoscope biopsy within the trachea or bronchus. Alternatively, the mouthpiece 20 can be a special purpose mouthpiece 20 that is used only during a replacement or change out ET tube procedure or other endoscopic procedure.

The cutting blade 30 can be configured to extend a short distance above the uppermost end of the short tube 20t, typically between 0.1 to about 0. 5 inches, more typically about 0.25 inches.

As shown in FIGS. 2A and 8A-8D, the short tube 20t can also or alternatively include an outer edge with at least one notch 23 such as a “V” shaped notch.

Where the mouthpiece 20 includes a biteblock 25 (FIGS. 2A, 8A-8D), it can be substantially rigid or may be flexible or semi-rigid for patient comfort. The biteblock 25 can have a straight length L₂ that is between about 1-2 inches (for adults) and a width W that is between about 1.5 to about 2 inches (for adults). As shown in FIGS. 2A, the biteblock 25 can have a radius of curvature that begins a distance inward of the external face-contacting surface 20s of the mouthpiece, after the straight segment, for a distance such as about 1-2 inches (for adults) past the straight segment.

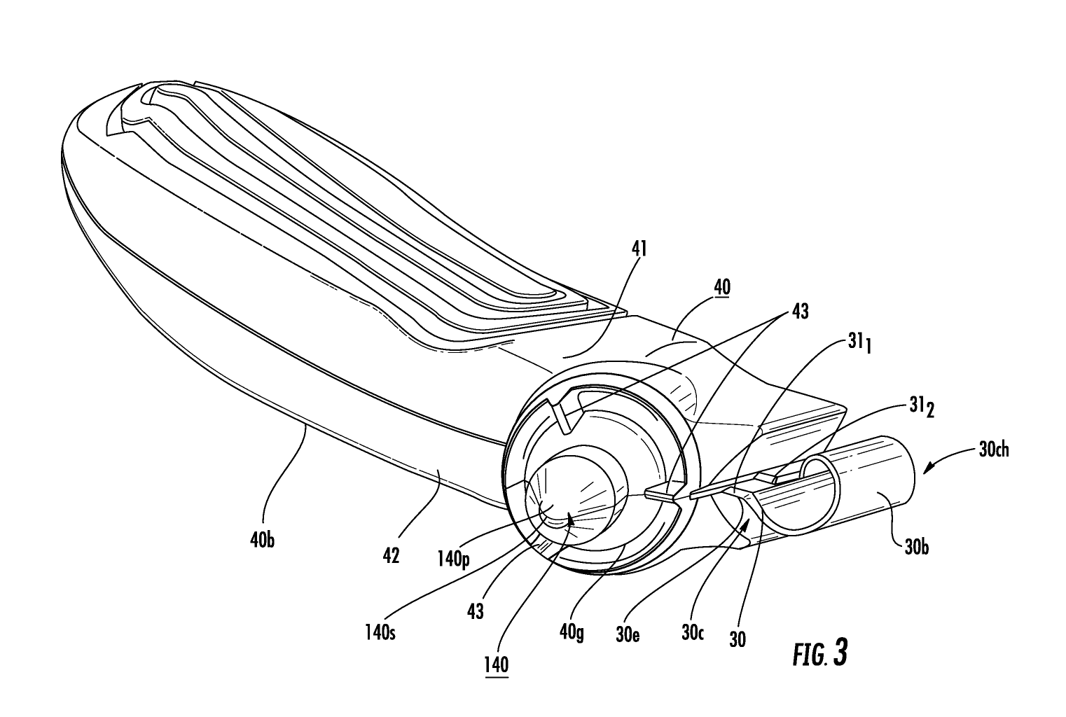

As shown in FIGS. 2A, 2E, 3-5, 6A, 6B and 7A-7C, the cutting blade 30 can comprise a shaped body that is configured to form a cutting edge 30e. The cutting blade 30 can comprise a band 30b of surgical grade metal, e.g., stainless steel, that can wrap around a particular size fiberoptic scope E. When wrapped around the scope E, the band 30b can tightly grasp the scope E. The band 30b can be preformed to have a curved shape to abut the endoscope outer wall and can define a substantially cylindrical channel 30ch (FIGS. 2B, 3). The band 30b can be formed to a curved shape in situ or prior to use (e.g., “pre-formed”). The band 30b can comprise a substantially rectangular-shaped body that can wrap about an axis, typically its long axis to form the cylindrical channel 30ch.

In some embodiments, the band 30b comprises a thin, sufficiently strong, malleable metallic material such as a metal shape-memory material. The band 30b can be provided pre-formed and sized for particular scopes. The band 30b can be formed in situ or on site corresponding to the scopes E at that facility and in use.

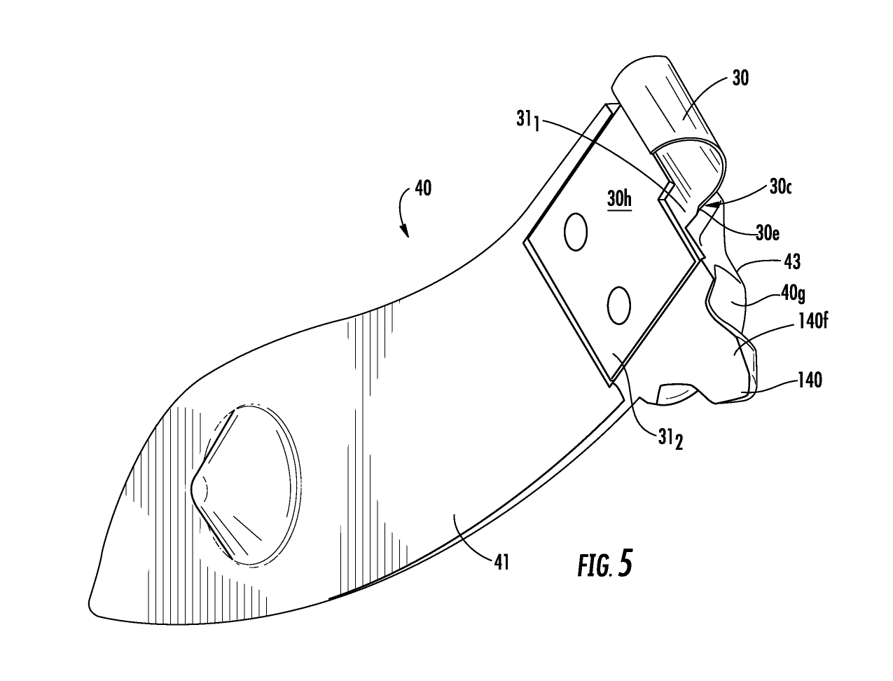

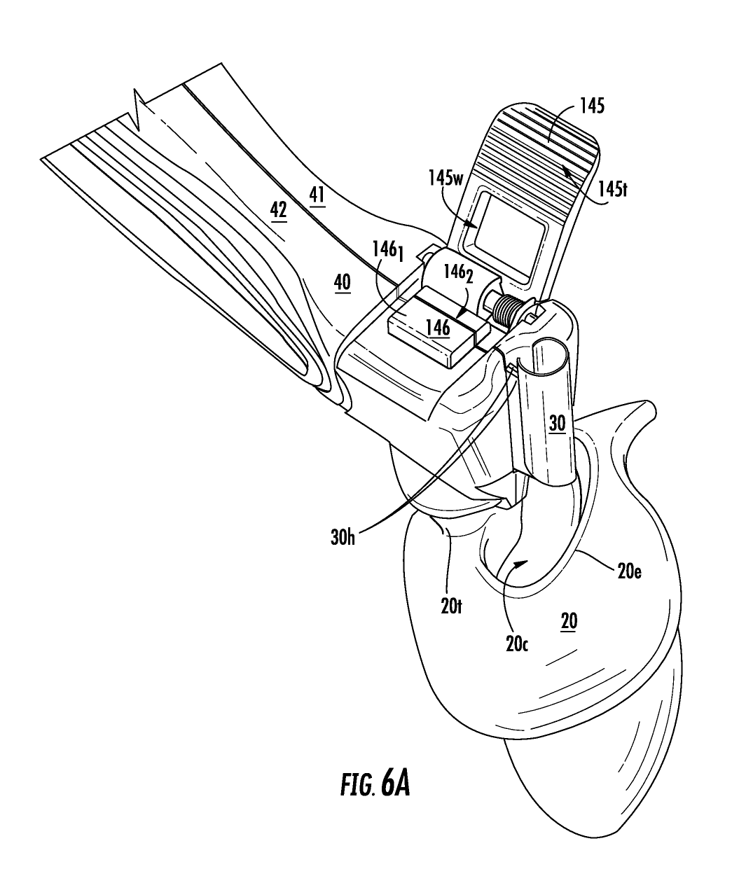

The band 30b can be configured to maintain a desired orientation/position of the cutting edge 30e by keeping the opposing flat portions 30h together during the cutting process. The band 30b can, in some embodiments, have a precision circumference to match a respective endoscope within about 0.5 mm, 1 mm or 2 mm. The band 30b can define a circle of a suitable (corresponding) diameter when the two opposed flat (straight) segments 30h take on a flat mating as shown in FIG. 6A and also allow the band 30b to open and the ends move apart without breaking.

The band 30b can also have other shapes. Non-cutting edges may be coated with a protective (spongy, foam-like material, rubber or other) material to inhibit user exposure to sharp edges.

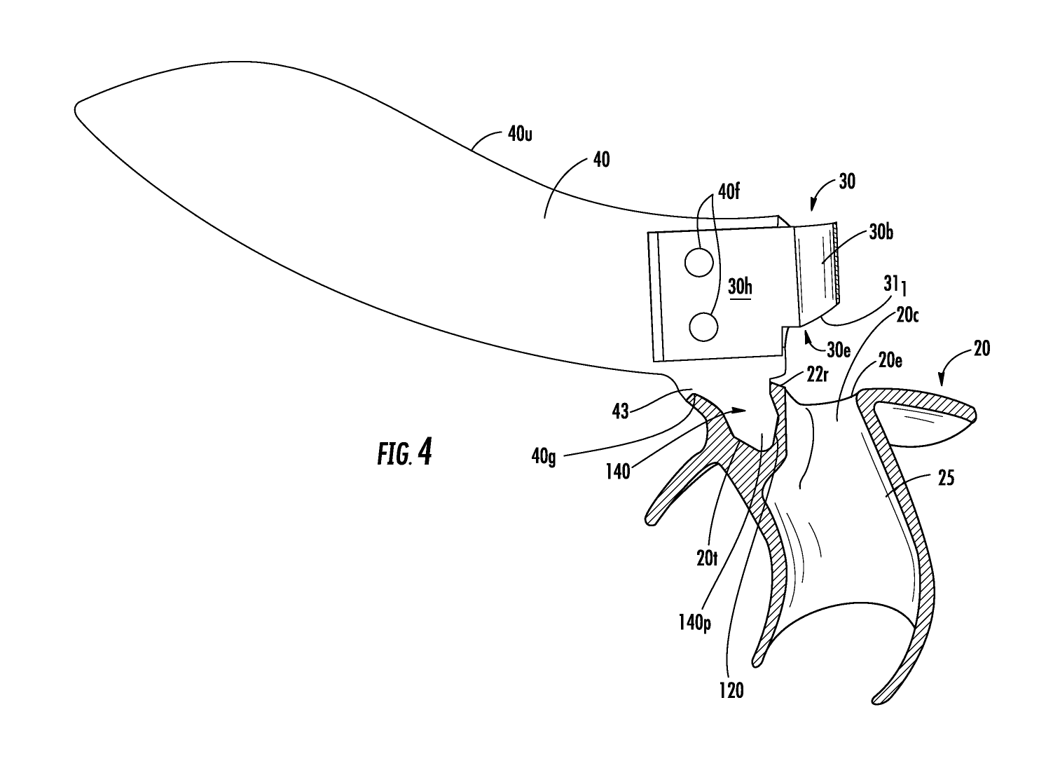

The cutting edge 30e of the band 30b can be a flat cutting edge surface 30c. The two long sides of the band 30b can have a length sufficient to extend above the cutting edge such as between about 10-40 mm, typically about 20 mm, with each side firmly anchored to each half of the handle 41, 42. As shown in FIGS. 3 and 4, for example, one long side of the band 30b can have a first segment 31₁ that extends a further distance down (relative to the orientation shown) than a second segment 31₂. The first segment 31₁ can define the flat cutting edge 30c/30e. The second segment 31₂. may also define a cutting edge as an alternative to or with the first segment 31₁.

The handle 40 or at least one of the handle members 41, 42 can be configured to have a releasable engagement with the cutting band 30b and can have secure-alignment and/or attachment features 40f with the band (e.g., male-female features, pins/holes and the like), so that a clinician can effect substantially instant or quick release of the two halves after cutting is complete. As shown in FIG. 4, the band 30b, when wrapped, includes at least one planar length 30h of fastening surface that attaches to at least one of the two matable handle members 41, 42.

Referring to FIGS. 4 and 5, for example, the distal inferior end of the cutting handle 40 can have groove 40g which snugly mates with a rim 22r of the short tube 20t. The upper part of the distal handle 40u that secures the cutting band 30b can be configured to closely approximate the cylindrical channel 30ch, nearly touching the scope E.

The cutting band 30b can be precision cut/sized according a corresponding (exact or within some tight tolerance) size (thickness/diameter) of the fiberoptic scopes E on hand at any institution. The cutting band 30b can accommodate a range of scopes E from thin to thick scopes (e.g., 5.3 mm, 5.7mm, 6.0 mm diameters). Alternately, model/size-specific bands 30b can be provided. The bands 30b can be provided in different sizes as a kit for selection onsite to match an endoscope in use.

The handle 40 can hold the cutting blade 30 in a closed or partially closed longitudinally extending (typically cylindrical) channel 30ch, and the handle 40 can interlock with the short tube 20t of the mouthpiece 20. The channel 30ch can be substantially cylindrical.

The short tube 20t that interlocks with the handle 40 can reside on the perimeter of the open channel 20c holding the ET tube 50 and is not required to receive the ET tube 50. Rather, the short tube 20t can be spaced apart from the ET tube open channel 20c of the mouthpiece 20 (i.e., and/or not defining an entry into the ET channel 20c and not holding the ET tube 50). It is noted that the term “interlock” and derivatives thereof means that the two components are physically attached to one another in a stable manner that can resist separation during normal intended use at least while compressive pressure/force is applied by a user, but does not require an actual locking relationship. The handle 40 and mouthpiece 20 can cooperate to provide an intuitive and easy-to-use arrangement to facilitate use without requiring laborious training.

As shown in FIGS. 2A-2E and 3, the handle 40 can have an interlocking segment 140 that resides in the short tube 20t to form a male/female interlocking attachment. The short tube 20t can include a cavity 120 for receiving the interlocking handle segment 140. The interlocking segment 140 can comprise a downwardly extending primary interlock member 140p that extends a distance below a primary bottom surface 40b of the handle 40 and can be snugly received into an interior cavity 120 (i.e., receptacle) of the short tube 20t.

The handle 40 can be configured to have a mechanical and/or visual guide for placing the handle 40 into the proper orientation into the mouthpiece 20. The attachment can be intuitive and easy-to use, even for first time users.

The short tube 20t can include guides/grooves and/or visual indicia of orientation and attachment features for facilitating proper interlocking and/or attachment to the handle 40.

Referring to FIGS. 4 and 8A-8D, the short tube 20t that engages the handle interlock segment 140 can have a closed cavity or an open cavity. The cavity 120 (and wall thereof) can taper inward from a larger outer size at the rim 22r to a smaller innermost size. The cavity 120 can be closed as shown in FIGS. 4 and 8A, or can be open or partially open. The cavity 120 can have a shape that corresponds to/matably receives the primary interlock member 140p. The primary interlock member 140p can have a length that is between about 0.25 inches and 4 inches, typically between about 0. 25 inches and about 1 inch, such as about 0.25 inches, about 0.3 inches, about 0.4 inches, about 0.5 inches, about 0.6 inches, about 0.7 inches, about 0.8 inches 0.9 inches and about 1 inch. The cavity 120 can have a depth that corresponds to the length of the primary interlock (projecting) member 140p, as shown in FIG. 4, or may be slightly larger. The cavity 120 can have a depth between about 0.25 inches and 4 inches, typically between about 0. 25 inches and about 1 inch, such as about 0.25 inches, about 0.3 inches, about 0.4 inches, about 0.5 inches, about 0.6 inches, about 0.7 inches, about 0.8 inches 0.9 inches and about 1 inch

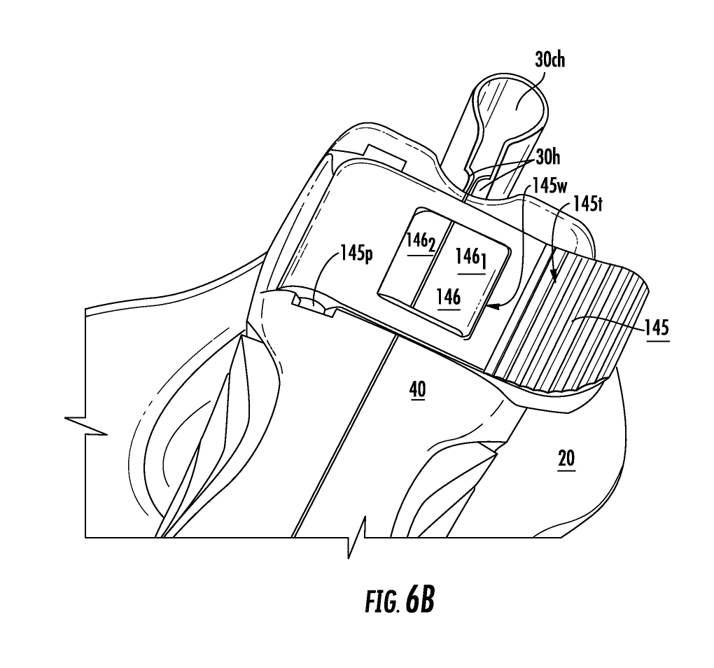

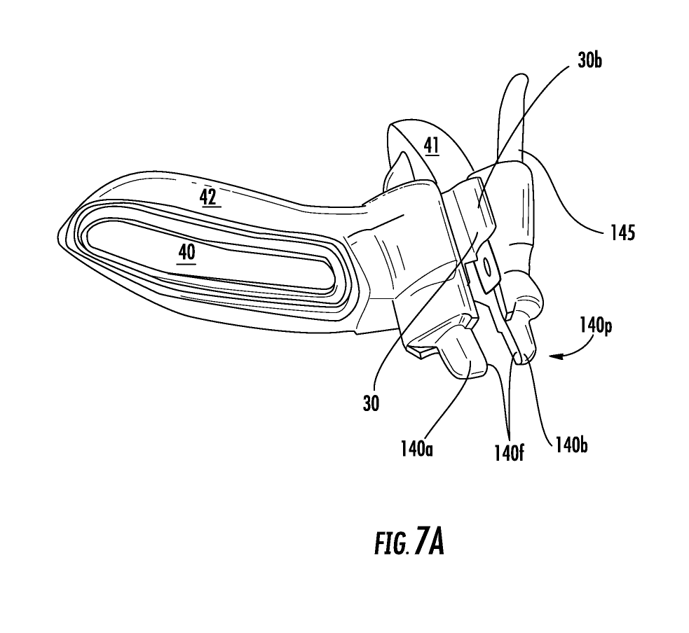

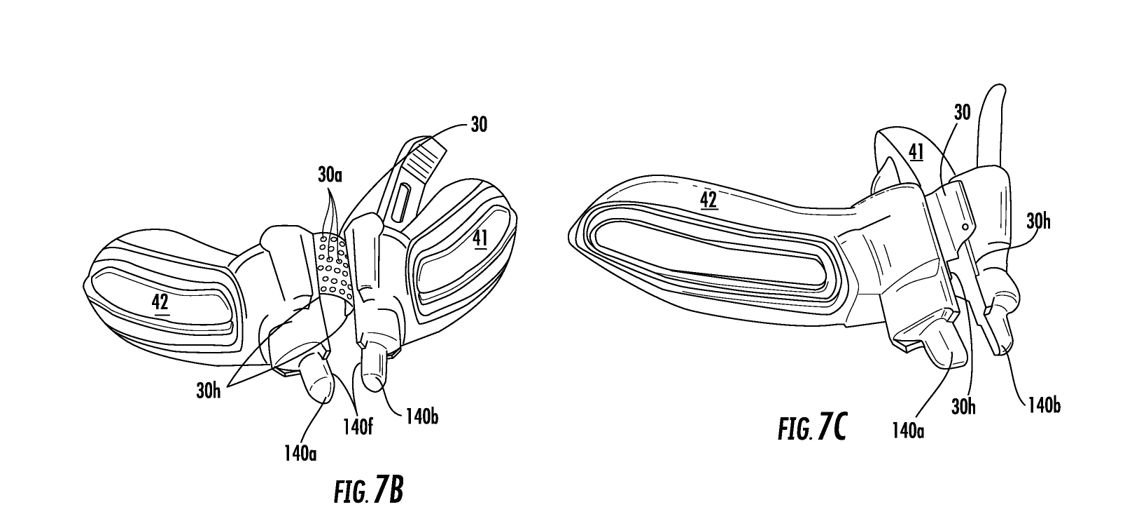

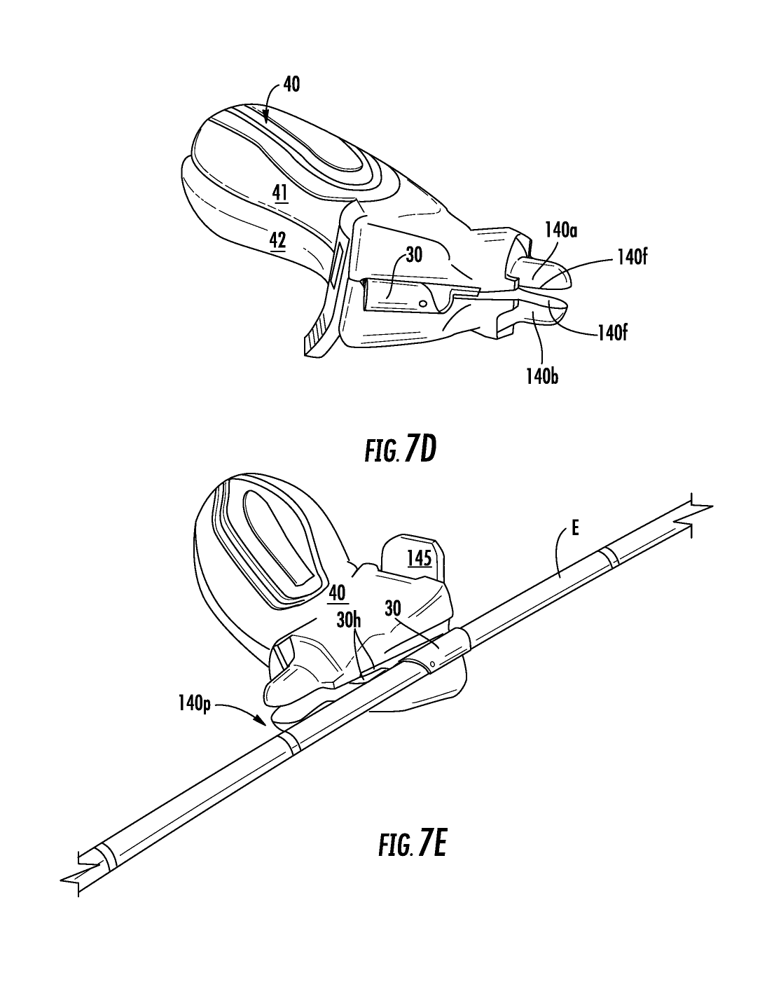

As shown in FIGS. 7A-7E, the first and second handle members 41, 42 can each hold a segment, e.g., in some embodiments a half, 140a, 140b of the primary interlock member 140p so that adjacent flat surfaces 140f can abut when in the cavity 120 of the short tube 20t and/or when the first and second handle members 41, 42 are held together. FIG. 3 illustrates the center split line 140s between the attached handle members 41, 42.

The primary interlock member 140 can have a hollow interior (FIG. 7D) or may be solid or partially hollow.

In some embodiments, as shown in FIG. 7D, when the handle members 41, 42 are latched together with latch 145 but prior to attachment to the cavity/receptacle 120 of the interlock channel of the short tube 20t, the pair of primary interlock members 140a, 140b can be spaced apart but proximate to each other (typically with respective flat surfaces facing each other). Entry into the receptacle 120 can force the members 140a, 140b to abut each other and interlock with the mouthpiece 20.

However, where a projecting interlock member 140 is used, it may be entirely held on one handle member (either 41 or 42) rather than partially on one and partially on the other where two cooperating handle members are used to form the handle.

FIGS. 2B and 3 illustrate that the end of the handle holding the cutting blade 30 can be arcuate.

The interlocking segment 140 can include a groove 40g that surrounds the primary interlock member 140p. As shown in FIG. 4, for example, in position, the groove 40g can rest against the rim 22r of the short tube 20t. However, other interlocking configurations of the mouthpiece 20 and handle 40 can be used. For example, the handle 40 can include a recess and the mouthpiece can have a protrusion, e.g., the short tube 20t can be solid and form the protrusion, and the handle 40 can have a recess that interlocks with the short tube 20t of the mouthpiece. In other examples, “press and rotate” or “press and twist”, and/or screw/threaded configurations may be used for interlocking the handle 40 and mouthpiece 20.

Referring to FIGS. 2A, 5 and 8A, for example, in some embodiments, the handle interlocking segment 140 can include a plurality of circumferentially spaced apart teeth 43 that can be received into respective notches 23 in the rim 22 of the short tube 20t. Although shown as three teeth 43, one, two or more than three teeth can be used. In other embodiments no teeth are used. The teeth 43 are shown as having “V” shapes that engage “V” shaped notches 23, but other teeth and notch shapes may be used.

Referring to FIG. 2B, the short tube 20t can cooperate with the interlocked handle 40 to position the cutting blade 30 over the open channel 20c of the mouthpiece 20. With the handle interlocking segment 140 in the short tube 20t, the centerline (C/L) of the longitudinally extending (e.g., cylindrical) channel 30ch can reside above and within the perimeter 20p of the external aperture/lumen/port 20e of the mouthpiece ET tube channel 20c. The handle 40 may be interlocked in either a right-hand or left-hand orientation on the mouthpiece 20. In FIG. 2B, the handle 40 can be held by a left hand of a user.

The band 30b can comprise a suitably sharp cutting surface 30e as shown in FIG. 5, for example. The band 30b can be metallic, typically surgical grade stainless steel, and may directly and snugly contact the endoscope (bronchoscope) during use. The band 30b can have internal surfaces that have increased friction such as foam, rubber or embossed surfaces and/or fenestrations 30a to firmly abut/secure the endoscope during a procedure (FIG. 7B).

Referring to FIGS. 2B, 4, and 8A-8D, for example, the open (ET tube) channel 20c can have an elongate external port/opening/aperture 20e that merges into the internal channel 20c containing the ET tube 50. As shown in FIG. 2B, the port/opening/aperture 20e can have a maximal width dimension “W” that is less than its maximal length dimension “L” and can merge into the channel of the bite block 25. The external opening/aperture 20e can be substantially oval or elliptical and allow the handle 40 to be used from a right or left side of a patient. The port 20e of the mouthpiece 20 can be a single, oval-shaped port that allows the “old” tube 50 to come out the right or left side. That is, the “old” ET tube 50 can be pulled to the left or right side of the mouthpiece 20 for removal and splitting based on sliding contact, upward against the cutting blade 30. The handle 40 can be interlocked to the mouthpiece 20 so that the cutting blade 30 faces the right or left side of the mouthpiece (or even the top or bottom side of the mouthpiece 20′, although not typical). The external channel port/aperture 20e can have a 35 mm×20 mm (L×W) dimension, in some embodiments.

The at least one short tube 20t can reside above but longitudinally outside the perimeter 20p of the external port 20e. The handle interlocking segment 140 can releasably interlock to a respective short tube 20t so that the cutting blade 30 extends inward from the short tube 20t to be positioned above but inside the perimeter 20p of the external port 20e.

Referring to FIGS. 8A-8D, the external aperture/opening 20e of the mouthpiece 20′ can have a perimeter shape 20p that is curvilinear and elongate in the length dimension “L”. The short tube 20t can reside medially, adjacent a bottom, caudal side 20b of the perimeter 20p. The external top, cephalad-wall 20w of the mouthpiece can be curved to comfortably fit under the nose of a patient. The short tube 20t can be configured with an upwardly extending wall 120w (in the orientation shown in FIG. 8B) that tapers to be larger at the top.

In some embodiments, the mouthpiece 20 can be a monolithic molded member and the inner wall 120w of the short tube 20t can be a bounding surface (wall) of a segment of the perimeter 20p, shown at a bottom perimeter, of the external port/opening/aperture 20e of the mouthpiece. In other embodiments, the short tube 20t can be positioned at other locations on the mouthpiece 20. However, the short tube 20t can be formed as a separate component that can be attached to the mouthpiece 20.

Further, while one short tube 20t is shown in FIGS. 8A-8D, two or more short tubes 20t may be used. Also, the location of the short tube 20t, whether single or a plurality, can be laterally and/or longitudinally offset from a center of the primary mouthpiece channel 20c.

FIGS. 6A and 6B illustrate that the handle 40 can include a pivotable latch or clasp 145 that is pivotably attached 145p to one of the cooperating first and second handle members 41, 42 to lock against a surface feature or latch member 146, such as a raised pad. The latch 145 can have a window 145w that closes against a surface feature or member on the adjacent handle member without the latch. The raised pad 146 can be configured as a pair of adjacent pads 146₁, 146₂, one on upper surface of each of the first and second handle members 41, 42. Once the old tube is removed, the clasp 146 can be released and the first and second handle members 41, 42 can be automatically pushed (spring) apart. While shown on the first handle member 41 to close when pushed to the left as shown in FIG. 6A, the latch can be configured to be pivotably attached to the right side member 42 and close by pivoting to the right over the other member 41.

As shown in FIG. 7A, for example, the cutting blade 30 can comprise a spring-steel cutting blade that “springs” laterally outward to open or partially open when the clasp 146 or other locking member is opened/unlocked/released. The clasp 145 can include a textured grip surface 145t. Thus, the handle members 41, 42 can be configured to spring apart once the clasp 146 is open for faster separation during a procedure to allow the new tube to be swiftly inserted over the bronchoscope with the cutting blade 30 out of the way.

At least one split location pin and/or other attachment member(s) can be used to align/join the first and second handle members 41, 42 for stability.

FIGS. 7A-7C illustrate exemplary handles 40 in an open or partially open/unlatched configuration. As shown, the handle members 41, 42 can be rotated apart as can the pair of interlock members 140a, 140b. The planar segments 30h of the metal cutting blade attached to respective handle members 41, 42 and can also separate from each other. Compare, for example, the adjacent, parallel (optionally abutting) configuration in FIGS. 7A, 7B and 7E with the spaced apart configuration shown in FIGS. 7B and 7C. The blade 30 can be pre-shaped/pre-formed to have a curved configuration when open/partially open.

FIG. 7B illustrates the metal band forming the cutting blade 30 with a fenestrated configuration, e.g., at least one aperture through the wall of the blade 30. The aperture(s) 30a can provide (additional) friction/grip features for the blade 30 to engage the shaft of the endoscope/bronchoscope E. The aperture(s) 30a can include a respective smooth perimeter segment(s) to allow a skin of the scope E to press into or through the aperture(s) 30a without damaging the outer scope membrane or material to provide (additional) friction/grip features for the cutting member 30 to engage the shaft of the endoscope/bronchoscope E. The apertures 30a can be regularly or irregularly spaced apart and may include 1-100 or more apertures of the same or different sizes and shapes.

The handle 40 can have various form factors and shapes. The handle 40 can have a lightweight but sufficiently rigid material to be able to provide the appropriate force to hold the endoscope E in position while the old tube 50 is pulled against the cutting blade 30. The handle 40 can comprise an ergonomic spring-foam and/or elastomeric or polymeric material. In some particular embodiments, the handle 40 can have maximal dimensions of 150 mm×35 mm×50 mm (lateral length/width/longitudinal length).

It is noted that some unexpected variation in endoscope (bronchoscope) diameter has been discovered. For example, two 5.7 diameter scopes have been found to have a 5.3 mm diameter. In some embodiments, the band 30b can have a 5.4 mm blade opening 30ch for 5.3 mm scope diameters. In use, actual measurements with a micrometer or a suitable metrology lab or OEM quality assurance certification of actual measurement for a respective scope may be appropriate for selecting blade sizes 30 for suitable channel diameters 30ch. It is also contemplated that the band 30b can be adjustable in diameter or to include surface features that can accommodate various different size scopes.

The band 30b can be sized and configured to provide a firm grip on the scope without crushing delicate fiber optics. The band 30b can be the grip member 35 described above or be used with another member as a cooperating grip member.

Thus, the device 10 can optionally also include a grip member that snugly attaches to the endoscope E to hold the endoscope E in position (and substantially if not totally stationary) in response to the holding force being applied while a clinician pulls the old tube 50old up to remove it from the patient. Where used, the grip member 35 can extend out from the handle 40d. The grip member can be a flexible (e.g. polymeric or rubber) strap with opposing sides held by distal end portions of the different handle members 41, 42.

The mouthpiece 20 can be a molded monolithic body with one or more integral short tubes 20t. Alternatively, other channels and channel members can be used. In some embodiments, a short tube 20 can be matably securely attached in situ to the primary mouthpiece body. The short tube 20t can threadably attach, adhesively attach or snap-fit into a recess/channel in the mouthpiece body, for example.

Embodiments of the invention can be carried out to maintain visualization (through the endoscope) of the carina (the distal end of the trachea that splits to right and left lung bronchi), while concurrently and also maintaining direct access within the trachea during the exchange process. This combination gives the clinician the reassurance that access, depth, stability, and pathway will not be compromised during the exchange.

Thus, the invention fills a long felt need that addresses the deficiencies and problems in conventional exchanges which could be frightening and dangerous, particularly in swollen or obese patients, or those with otherwise difficult intubations. In the past, typically, the old tube must be pulled out blindly over an exchange rod, tube, stylette or bougie, and then the new tube is slid over the bougie blindly and without stability. One may lose access to the trachea as the rod, tube or bougie, inadvertently, slides in/out, or kinks into the esophagus, or it may go in too far and puncture the bronchus, or might go through the ET tube Murphy eye.

Thus, the methods and devices contemplated by embodiments of the invention are configured so that the pathway is not lost and the sight of the pathway inside the trachea during the exchange is maintained.

It is contemplated that such tubes can be changed out as needed for malfunction or for size change, or on any schedule deemed appropriate by medical care.

In some embodiments, such as where the mouthpiece is a “special purpose” mouthpiece used for the change out, the procedure can be carried out as follows. The mouthpiece 20 is put in place (threaded or slid over) the indwelling ET tube 50, after removing the ET adaptor, allowing it to protrude from one of the holes 20c (allowing a user to select, as there are typically two apertures/channels to accommodate left/right handed users and/or to allow the procedure to be done from the other side of the patient). A new ET tube is (previously) preloaded onto a long endoscope. Then, the handle 40 with cutting blade 30 (typically of formed band 30b) can be wrapped around the shaft of the bronchoscope distal to the new ET tube.

Then the old ET tube 50old is grabbed with forceps or a clamp 310, after removal of the ET tube adaptor. The endoscope E is then pushed through the old indwelling ET tube (so that the endoscope has both the new and old ET tubes on it). The handle 40 can be anchored to the mouthpiece short tube 20t. The anchoring may be via the interlock segment 140 such as using projection member 140p and/or groove 40g at the upper rim 22r. Then the old ET tube 50old is pulled up. As it is pulled up, it is slit open longitudinally by the cutting blade 30, so that the old ET tube peels off the endoscope E to the side. During this time, the endoscope is still securely in the patient's airway providing direct continuous visualization of the carina by the clinician. Then, the handle 40 is removed and the new ET tube 50new (which has been at the top of the endoscope this whole time) is slid down the endoscope E and into the airway, as it would be during any typical and common fiberoptic intubation.

It is noted that the handle 40 may also hold a grip, such as a curved or cylindrical shaped elastomeric grip, that can be positioned with the wrapped band 30b (inside, below or above the band) while the cutting blade 30 contacts the old ET tube 50old.

The handle 40 and mouthpiece 20 can be used to rapidly remove and replace an endotracheal tube (typically in under about 1 minute). The patient can be disconnected from a ventilator during the pulling and cutting steps, then reconnected after the inserting step.

Referring now to FIG. 8, a set of surgical tools can be provided as a kit 300 in a sterile package 300p. The kit 300 includes a clamp 310, such as a Kocher clamp, hemostat or forceps or toothed clamp, and the handle 40 with the cutting blade 30. The kit may optionally include a surgical knife blade (e.g., a #11 surgical knife blade) as a back-up or supplemental cutting device.

Optionally, the mouthpiece 20 may also be included in the kit 300.

The kit 300 can be provided with the cutting blade 30 pre-attached to the handle 40 and ready to use. Alternatively, different size blades 30 with a pre-formed curved segment of a defined radius and/or diameter may be included in a single kit for user selection in situ.

It is noted that the handle 40 may be suitable for use for other procedures and is not limited to the embodiments discussed specifically herein. Similarly, the mouthpiece 20 may be suitable for facilitating other procedures and is not limited to the ET tube changeout described hereinabove.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1. A tracheal tube device, comprising:
a mouthpiece comprising an external port merging into an open internal channel sized and configured to allow an endotracheal tube to extend therethrough into a trachea of a subject, the external port having a perimeter, wherein the mouthpiece also comprises at least one short tube with a cavity residing above the external port, and wherein an outerwall of at least one of the at least one short tube defines a bounding surface of the perimeter of the external port, and/or wherein at least one of the at least one short tube resides adjacent a medial bottom segment of the external port; and
a handle with at least one interlock member, wherein, in an operative configuration, the handle extends laterally away from the mouthpiece to position a distal end thereof at between 3-10 inches away from the mouthpiece, wherein the interlock member interlocks to the at least one short tube and comprises a cutting blade that extends inward from a respective short tube to be positioned above but inside the perimeter of the external port.

2. The device of claim 1, wherein the external port has an elongate configuration with a width dimension greater than a length dimension, wherein the short tube resides proximate a medial segment of the width dimension, and wherein, in use, the handle can be held by the short tube to be able to selectively and laterally extend to either a right or left side of a patient or in a caudal orientation to the patient.

3. The device of claim 1, wherein the short tube resides adjacent the medial bottom segment of the external port.

4. The device of claim 1, wherein the at least one interlock member comprises a primary interlock member that projects a distance of between 0.25 inches and 4 inches below a bottom surface of the handle and is slidably receivable into the cavity of the short tube, and wherein the cavity of the short tube has a depth that is between about 0.25 inches and 4 inches.

5. The device of claim 1 wherein the short tube has an outwardly extending wall that tapers inwardly in a direction toward the external port of the mouthpiece into a smaller size.

6. The device of claim 5, wherein the primary interlock member is provided as first and second cooperating downwardly extending interlock members, the first interlock member attached to the first handle member and the second interlock member attached to the second handle member.

7. The device of claim 6, wherein the first and second interlock members are semicircular member with flat inner surfaces that face each other.

8. The device of claim 1, wherein the handle holds a metallic band defining the cutting blade, wherein the metallic band has a substantially cylindrical longitudinally extending segment, wherein, in an operative configuration, the handle extends laterally away from the mouthpiece while the at least one interlock member resides in the short tube with the cylindrical segment held over the external port of the mouthpiece a distance in front of the short tube.

9. The device of claim 8, wherein the substantially cylindrical segment has a fenestrated configuration.

10. The device of claim 1, wherein the handle comprises:
first and second cooperating handle members that can open and close relative to each other and that hold a metallic member defining the cutting blade therebetween, and
a pivotable latch member residing on one of the first and second members configured to latch and unlatch the first and second members.

11. The device of claim 10, wherein the first and second handle members comprise an outer upper surface, each with aligned raised pads, and wherein the latch comprises a window that encases the aligned raised pads in a latched configuration.

12. The device of claim 1, wherein the at least one short tube is a single short tube, wherein the short tube has an outer rim with notches, wherein the at least one handle interlock member comprises a lower surface with a circumferentially extending groove and radially extending teeth, and wherein the groove is configured to abut the rim of the short tube and the teeth are configured to reside in respective notches.

13. The device of claim 12, wherein the notches have an open “V” shape with a larger end facing upward and the teeth have a V shape with a smaller end facing downward.

14. The device of claim 1, wherein the cutting blade is configured as a malleable unitary surgical metallic band that wraps together to define a cylindrical channel and defines a cutting edge.

15. A medical mouthpiece, comprising:
a mouthpiece comprising an external port merging into an open internal channel sized and configured to allow an endotracheal tube to extend therethrough, the external port having a perimeter, wherein the mouthpiece also comprises at least one short tube with a cavity residing above the external port, wherein the mouthpiece has a monolithic molded body, and wherein an outerwall of at least one of the at least one short tube defines a bounding surface of the perimeter of the external port and/or at least one of the at least one short tube resides adjacent a medial bottom segment of the external port.

16. The mouthpiece of claim 15, wherein the external port has an elongate configuration with a width dimension greater than a length dimension, wherein the short tube resides proximate a medial segment of the width dimension.

17. The mouthpiece of claim 15, wherein the cavity of the short tube has a depth that is between about 0.25 inches and 4 inches.

18. The mouthpiece of claim 15, wherein the short tube has an outwardly extending wall that tapers inwardly in a direction toward the external port of the mouthpiece into a smaller size.

19. The mouthpiece of claim 15, wherein the at least one short tube is a single short tube, wherein the short tube has an outer rim with circumferentially spaced apart notches.

20. A medical device, comprising:
a handle comprising first and second cooperating, laterally extending handle members with a length between 3-10 inches that can open and close relative to each other, the handle holding a metallic band defining a cutting blade therebetween, wherein the metallic band has a cylindrical segment that resides outside the first and second handle members, and wherein the handle comprises an interlock member that projects a distance of between 0.25 inches and 4 inches below the handle.

21. The device of claim 20, further comprising a pivotable latch member residing on one of the first and second members configured to latch and unlatch the first and second members.

22. The device of claim 20, wherein the interlock member is provided as first and second cooperating semicircular halves, a first half attached to the first handle member and the second half attached to the second handle member.

23. The device of claim 20, wherein each of the cooperating semicircular halves have flat inner surfaces that face each other.

24. The device of claim 21, wherein the first and second handle members comprise an outer upper surface, each with aligned raised pads, and wherein the latch comprises a window that encases the aligned raised pads in the latched configuration.

25. The device of claim 20, wherein the handle comprises a lower surface with a circumferentially extending groove extending about the interlock member and radially extending teeth that extend inward from the groove.

26. The device of claim 25, wherein the teeth have a V shape with a smaller end facing downward.

27. The device of claim 20, wherein the metallic band has a fenestrated configuration.

28. (canceled)
